(19) United States
(12) Patent Application Publication
(10) Pub. No.: US 2015/0051630 A1
(43) Pub. Date: Feb. 19, 2015

(54) LANCING DEVICE WITH VIBRATION ABSORPTION MECHANISM

(71) Applicant: BIOTEST MEDICAL CORPORATION, Taichung City (TW)

(72) Inventor: Chen-Heng CHENG, Taichung City (TW)

(73) Assignee: BIOTEST MEDICAL CORPORATION, Taichung City (TW)

(21) Appl. No.: 14/029,044

(22) Filed: Sep. 17, 2013

(30) Foreign Application Priority Data
Aug. 19, 2013 (TW) 102129706

(51) Int. Cl. A61B 5/15; A61B 5/151

(52) U.S. Cl. CPC A61B 5/1411; A61B 5/15101
USPC 606/181

(57) ABSTRACT

A lancing device includes a main body and a replaceable lancet. A front cover is detachably mounted to a front end of the main body and formed by a shell and a positioning member connected inside the shell for receiving the lancet. A shock absorber is mounted in between the shell and the positioning member. When the lancet is fired by a built-in percussion unit of the main body to move forward, the front end of the lancet collides with a stop portion of the positioning member, and the lancet stops movement and instantaneously retracts. Meanwhile, most or all of the vibrational energy generated when the front end of the lancet collides with the positioning member can be absorbed by the shock absorber. In this way, the vibration in the process of the blood sampling can be reduced to further minimize the pain felt by the testee.

### BACKGROUND OF THE INVENTION

1. Field of the Invention

The present invention relates generally to a blood-sampling device and more particularly, to a shock-absorbable lancing device which can reduce the vibration generated while a lancet penetrates through and retracts from the human skin to make a testee feel less pain.

2. Description of the Related Art

When a diabetic or chronic patient needs a blood test, the medical personnel usually use a lancet firing from a lancing device to penetrate through a testee's skin to make it bleed a little, and the little blood is collected for test. A conventional lancing device usually includes a pen-shaped housing, a replaceable lancet, and a front cover detachably mounted to the housing for installing the lancet. The lancet includes a needle mounted to a lancet holder inside the housing. While the lancet holder is moved to a predetermined position, the needle is exserted out of the front cover to penetrate through the testee's skin for predetermined depth and then quickly retracts.

From the perspective of the testee, the quicker the blood sampling process is accomplished, the less psychological burden and terror the testee will have. Thus, the testee would normally press the trigger button of the percussion unit of the conventional lancing device to promptly fire the lancet, making the lancet to move quickly to protrude out of the front cap. However, besides the painful feeling the testee usually has while the lancet penetrates through the skin at high speed, the testee will feel even more pain and the pierced wound would expand as well while the lancet reaches the deepest below the skin as it could, where the maximum vibration is generated from the collision and retraction of the lancet.

### SUMMARY OF THE INVENTION

The primary objective of the present invention is to provide a lancing device having a shock absorber, which can absorb the vibration generated at the moment that the lancet thereof penetrates through the testee's skin and quickly retracts to effectively relieve the testee's pain and to prevent the testee wound from expanding.

The foregoing objective of the present invention is attained by the lancing device formed of a main body and a front cover mounted to a front end of the main body. The front cover can be in the form of a shell. The lancing device includes an extended hollow positioning member sleeved into the shell, a lancet movably mounted inside the positioning member, and a shock absorber mounted in between the shell and the positioning member. In light of the structure mentioned above, when the lancing device is operated for the blood sampling process, the kinetic energy of the lancet can be transferred to the positioning member and the shock absorber can serve as a damper to provide proper resilience and damping, based on the vibration generated in the process of the movement, and vibration generated by the immediate retraction of the lancet can be reduced to further reduce the pain that is felt by the testee.

In a preferred embodiment of the present invention, the shock absorber is a hollow elastic member or an extended sleeve mounted around the positioning member and made of rubber, a thermoplastic material, or other resilient materials.

### BRIEF DESCRIPTION OF THE DRAWINGS

FIG. 1 is a side view of a first preferred embodiment of the present invention.

FIG. 2 is a partially sectional view of the first preferred embodiment of the present invention.

FIG. 3A is another partially sectional view of the first preferred embodiment of the present invention, illustrating that the needle of the lancet protrudes from the main body.

FIG. 3B is an enlarged view of a part of the shock-absorbable lancing device shown in FIG. 3A.

FIG. 4 is a partially sectional view of a second preferred embodiment of the present invention.

### DETAILED DESCRIPTION OF PREFERRED EMBODIMENTS

Structural features and desired effects of the present invention will become more fully understood by reference to embodiments given hereunder. However, it is to be understood that these embodiments are given by way of illustration only, thus are not limitative of the claim scope of the present invention.

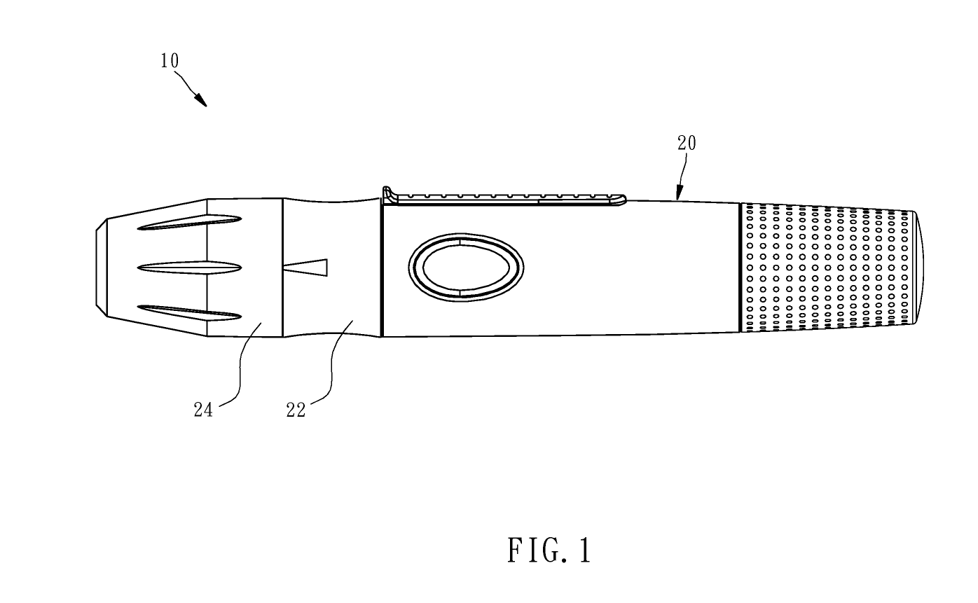

Referring to FIGS. 1, 2, 3A, and 3B, a lancing device 10, constructed according to a first embodiment of the present invention, is formed of a main body 20, a lancet 30, and a shock absorber 40. The detailed descriptions and operations of these elements as well as their interrelations are recited in the respective paragraphs as follows. In addition, please note that the directions mentioned hereinafter are defined based on the cognizance of an ordinary person, e.g., the lancet 30 protrudes from the main body 20 at the front side of the lancing device 10.

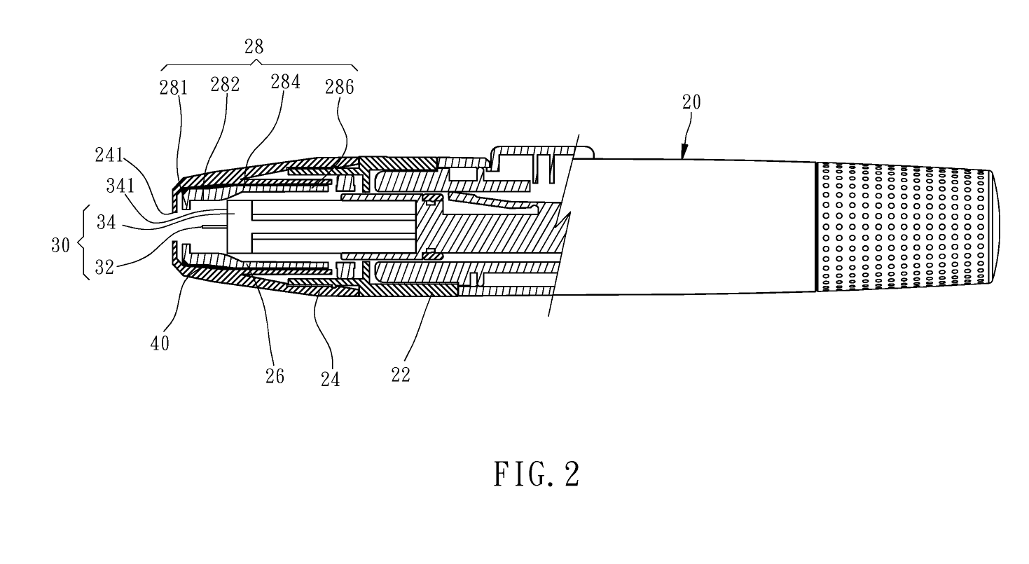

The main body 20 is pen-shaped and includes a body portion 22 and a front cover detachably mounted to a front end of the body portion. As shown in FIG. 2, the front cover includes a shell 24 sleeved to a fastening seat, and a positioning member 26 coaxially connected inside the shell 24. An outlet 241 is formed at a front end of the shell 24. A slide channel 28 is formed inside the positioning member 26 and communicates with the outlet 241. The outlet 241 and the slide channel 28 are coaxially arranged. Specifically, the slide channel 28 is circular in cross-sectional view and includes a stop portion 281, a small-diameter portion 282, a taper-shaped portion 284, and a large-diameter portion 286 in turn toward a direction away from the outlet 241. The small-diameter portion 282 is smaller than the large-diameter portion 286 and the stop portion 281 is smaller than the small-diameter portion 282.

The lancet 30 is a replaceable part and includes a needle 32 and a needle holder 34 slidably mounted inside the slide channel 28. The needle holder 34 can be driven to move forward and backward by a built-in percussion unit located inside the main body 20.

The shock absorber 40 is a hollow resilient member made of rubber or a thermoplastic material, surrounding the positioning member 26 and covering an external periphery of the positioning member 26 to make the positioning member 26 be tightly fitted with the shell 24 to further effectively position the lancet 30 inside the positioning member 26. In addition, the shock absorber 40 can provide uniform and balanced shock absorption while the lancet 30 is moving. It is worth mentioning that the hollow resilient member can be integrally formed on the positioning member 26 by injection molding.

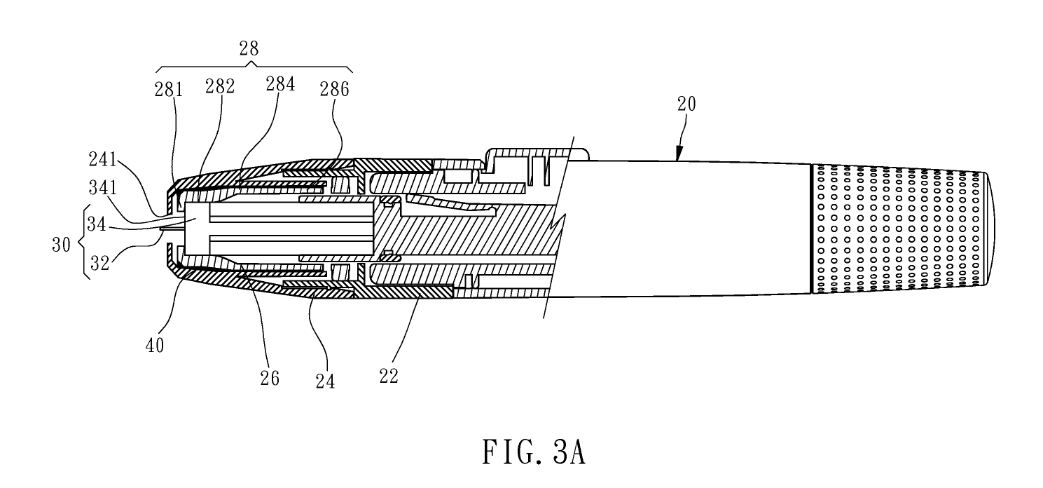

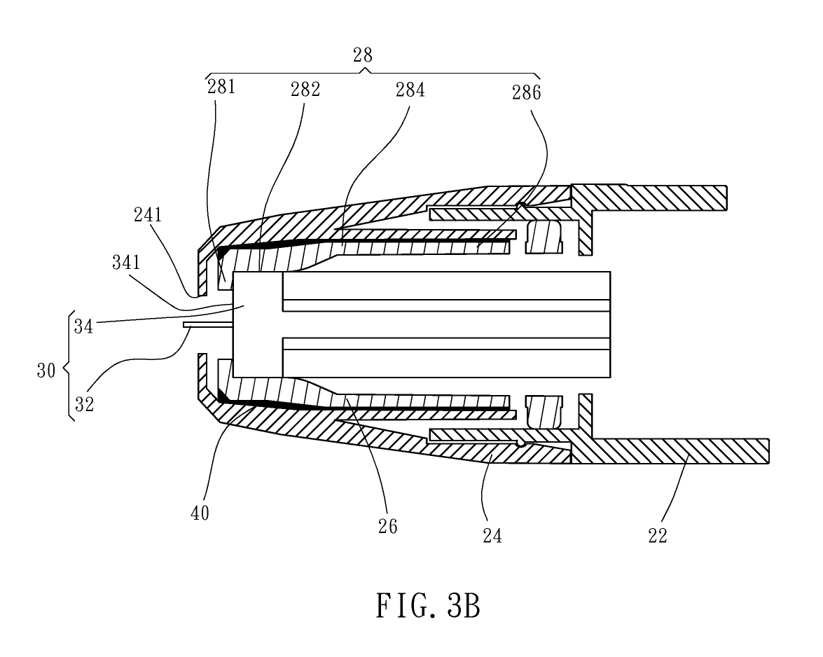

Referring to FIGS. 2, 3A, and 3B, when the testee operates the percussion unit to drive the lancet 30 to move toward the outlet 241, a front portion 341 of the needle holder 34 passes through the large-diameter portion 286 of the slide channel 28 and then is guided by the taper-shaped portion 284 to enter the small-diameter portion 282. Next, the lancet 30 continues to axially move along the small-diameter portion 282 toward the outlet 241, and finally the front portion 341 contacts against the stop portion 281 to stop movement. In the meantime, the needle 32 protrudes from the outlet 241 to further penetrate through the testee's skin and to reach the deepest below the skin as it could. In the process of the movement of the lancet 30, the needle holder 32 will be guided by the slide channel 28, so some vibration and kinetic energy generated by the lancet 30 in radial direction will be transmitted to the positioning member 26 and finally absorbed by the shock absorber 40. While the lancet 30 moves forward to make the front portion 341 contact the stop portion 281, the positioning member 26 can stop the lancet 30 from moving forward to result in a collision between the stop portion 281 of the positioning member 26 and the lancet 30. Because the action force and the reaction force of the positioning member 26 and the lancet 30 generates great vibrational energy and because the vibrational energy can be absorbed by the shock absorber 40, the vibration of the lancet 30 can be greatly reduced to produce a smooth movement of the lancet 30, which further reduces the pain that is felt by the testee. In addition, the vibrational energy generated by another element(s) of the lancing device 10, for example, the built-in percussion unit, will be absorbed by the shock absorber 40 to further reduce the testee's pain.

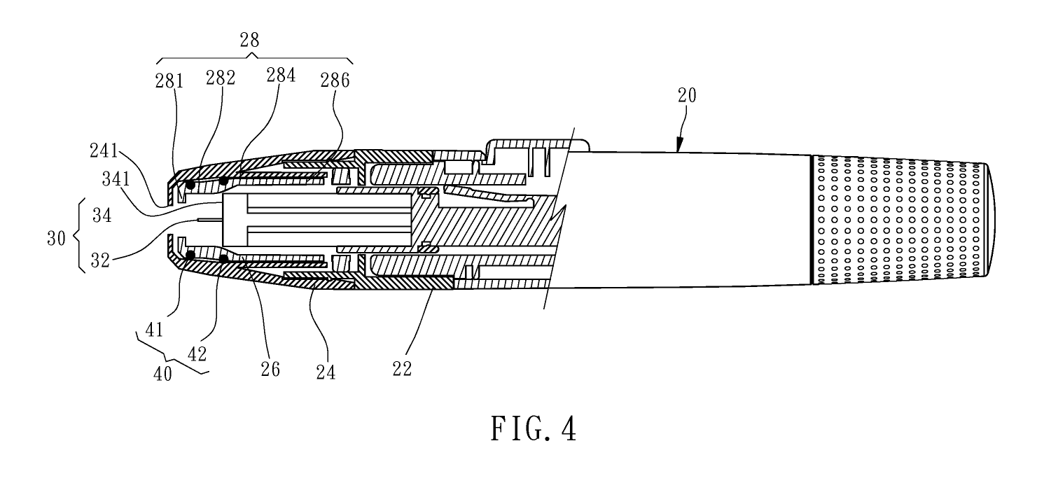

Referring to FIG. 4, a shock-absorbable lacing device 10 is constructed according to another embodiment of the present invention. In an embodiment, the shock absorber 40 is formed of two or more rubber rings 41 and 42 spaced from each other and sleeved onto a front section and a rear section of the positioning member 26. Likewise, the two rubber rings 41 and 42 can provide superior shock absorption.

It is worth mentioning that when the needle holder 34 moves to the small-diameter 282 portion of the positioning unit 26, the vibration particularly tends to occur because the small-diameter portion 282 has almost the same area of the cross-section as that of the needle holder 34 and the needle 32 is about to contact the testee's skin. In an embodiment, the shock absorber 40 can only include one rubber ring which can be mounted to the front section of the positioning member 26 to absorb the vibrations and to further make the needle 32 smoothly protrude from the outlet 241, so the vibration generated by the collision can be reduced to relieve the pain felt by the testee.

In conclusion, the shock absorber of the present invention can absorb the mechanical energy generated by the rapid movement and collision of the lancet to reduce the vibration of the lancet inside the human body. The technician in the art can optionally adopt either of structures and either of materials for the shock absorber to get a preferable damping value as it depends. For example, the shock absorber can be resilient columns arranged annularly for providing the same shock-absorbable effect. Therefore, the present invention can definitely minimize the pain that the testee suffers to facilitate the blood sampling process.

What is claimed is:

1. A lancing device comprising a main body and a front cover mounted to a front end of the main body, the front cover being formed of a shell and a positioning member connected inside the shell, a lancet being slidably mounted inside the positioning member, the lancing device being characterized in that a shock absorber mounted in-between the shell and the positioning member.

2. The lancing device as defined in claim 1, wherein the shock absorber surrounds the positioning member.

3. The lancing device as defined in claim 2, wherein the shock absorber is a hollow resilient member.

4. The lancing device as defined in claim 3, wherein the hollow resilient member is made of rubber or a thermoplastic flexible material.

5. The lancing device as defined in claim 3, wherein the shock absorber comprises at least one rubber ring mounted to an external periphery of a front section of the positioning member along the moving direction of the lancet.

6. The lancing device as defined in claim 5, wherein the at least one rubber ring is two in number and the two rubber rings are spaced from each other, the other rubber ring being mounted to an external periphery of a rear section of the positioning member along the moving direction of the lancet.

7. The lancing device as defined in claim 3, wherein the hollow resilient member covers the external periphery of the positioning member.

8. The lancing device as defined in claim 7, wherein the hollow resilient member is integrally formed on the positioning member.

9. The lancing device as defined in claim 1, wherein the shell and the positioning member are coaxially arranged, the shell having an outlet, the positioning member having a slide channel formed therein and communicating with the outlet, the slide channel having a small-diameter portion, a taper-shaped portion, and a large-diameter portion in turn toward a direction away from the outlet.

10. The lancing device as defined in claim 2, wherein the shell and the positioning member are coaxially arranged, the shell having an outlet, the positioning member having a slide channel formed therein and communicating with the outlet, the slide channel having a small-diameter portion, a taper-shaped portion, and a large-diameter portion in turn toward a direction away from the outlet.

11. The lancing device as defined in claim 3, wherein the shell and the positioning member are coaxially arranged, the shell having an outlet, the positioning member having a slide channel formed therein and communicating with the outlet, the slide channel having a small-diameter portion, a taper-shaped portion, and a large-diameter portion in turn toward a direction away from the outlet.

12. The lancing device as defined in claim 4, wherein the shell and the positioning member are coaxially arranged, the shell having an outlet, the positioning member having a slide channel formed therein and communicating with the outlet, the slide channel having a small-diameter portion, a taper-shaped portion, and a large-diameter portion in turn toward a direction away from the outlet.

13. The lancing device as defined in claim 5, wherein the shell and the positioning member are coaxially arranged, the shell having an outlet, the positioning member having a slide channel formed therein and communicating with the outlet, the slide channel having a small-diameter portion, a taper-shaped portion, and a large-diameter portion in turn toward a direction away from the outlet.

14. The lancing device as defined in claim 6, wherein the shell and the positioning member are coaxially arranged, the shell having an outlet, the positioning member having a slide channel formed therein and communicating with the outlet, the slide channel having a small-diameter portion, a taper-shaped portion, and a large-diameter portion in turn toward a direction away from the outlet.

15. The lancing device as defined in claim 7, wherein the shell and the positioning member are coaxially arranged, the shell having an outlet, the positioning member having a slide channel formed therein and communicating with the outlet, the slide channel having a small-diameter portion, a taper-shaped portion, and a large-diameter portion in turn toward a direction away from the outlet.

16. The lancing device as defined in claim 8, wherein the shell and the positioning member are coaxially arranged, the shell having an outlet, the positioning member having a slide channel formed therein and communicating with the outlet, the slide channel having a small-diameter portion, a taper-shaped portion, and a large-diameter portion in turn toward a direction away from the outlet.
